(19) United States
(12) Patent Application Publication
(10) Pub. No.: US 2009/0271882 A1
(43) Pub. Date: Oct. 29, 2009

(54) Transgenic Pig with Diabetes and Method for Producing the Same

(75) Inventors: Kazuhiro Umeyama, Kanagawa (JP); Hiroshi Nagashima, Kawasaki-shi (JP); Masahito Watanabe, Fujisawa-shi (JP); Keizaburo Miki, Kanagawa (JP)

Correspondence Address: BIRCH STEWART KOLASCH & BIRCH, PO BOX 747, FALLS CHURCH, VA, 22040-0747, US

(73) Assignee: Bios Research Institute Inc., kanagawa (JP)

(21) Appl. No.: 11/992,317

(22) PCT Filed: Aug. 24, 2006
(86) PCT No.: PCT/JP2006/316581

§ 371 Date: Sep. 2, 2008

(30) Foreign Application Priority Data
Sep. 21, 2005 (JP) 2005 273997

(51) Int. Cl. A01K 67/027; A01K 67/02

(52) U.S. Cl. 800/17; 800/25

(57) ABSTRACT

A transgenic animal having diabetes, which is more suitable as a model of human than rodents, and its preparation method are disclosed. The method for preparing the transgenic pig comprises introducing a nucleic acid into a fertilized egg, clonal egg or embryo, the nucleic acid comprising a foreign gene which contains a region encoding dimerization domain of hepatocyte nuclear factor-1α, but does not encode a normal hepatocyte nuclear factor-1α, and a promoter located upstream of the foreign gene, which promoter is capable of expressing the foreign gene in a pig cell; and developing an individual from the fertilized egg, clonal egg or embryo.

### TECHNICAL FIELD

The present invention relates to a transgenic pig with diabetes and method for producing the same.

### BACKGROUND ART

Hepatocyte nuclear factor (HNF) is a factor participating in transcriptional regulation, and HNF-1α, HNF-1β, HNF-4α and so on are known. If HNF-1α is mutated, transcriptional regulation cannot be accomplished. As a result, expression of insulin gene, glucose transporter 2 gene and glucokinase gene becomes insufficient, and growth of pancreatic β cells also becomes insufficient, so that diabetes appears. In case of maturity-onset diabetes of youth (MODY) which occupies 2 to 3% of total diabetes, diabetes appears through autosomal dominant inheritance, and 6 causative genes have been identified so far. Among them, abnormality of HNF-1α has been identified as the causative gene of MODY3, and the frequency thereof is the highest in MODYs among Japanese people. Preparation of transgenic mice having diabetes by introducing HNF-1α P291fsinsC which is a mutated gene of HNF-1α has been reported by two groups.

Non-patent Literature 1: Endocrinology Vol. 142, 5311-5320, 2001 KERSTIN A. HAGENFELDT-JOHANSSON et al. β-Cell-Targeted Expression of a Dominant-Negative Hepatocyte Nuclear Factor-1α Induces a Maturity-Onset Diabetes of the Young(MODY)3-Like Phenotype in Transgenic Mice.
Non-patent Literature 2: Diabetes Vol 51, 114-123, 2002 Kazuya Yamagata et al. Overexpression of Dominant-Negative Mutant Hepatocyte Nuclear Factor-1α in Pancreatic β-Cells Causes Abnormal Islet Architecture With Decreased Expression of E-Cadherin, Reduced β-cell Proliferation, and Diabetes.
Non-patent Literature 3: Kurome M, Fujimura T, Murakami H, Takahagi Y, Wako N, Ochiai T, Miyazaki K, Nagashima H. Comparison of electro-fusion and intracytoplasmic nuclear injection methods in pig cloning. Cloning and Stem Cells 2003; 5: 367-378
Non-patent Literature 4: Kurihara T, Kurome M, Wako N, Ochiai T, Mizuno K, Fujimura T, Takahagi Y, Murakami H, Kano K, Miyagawa S, Shirakura R, Nagashima H. Developmental competence of in vitro matured porcine oocyte after electrical activation. J Reprod Dev 2002; 48: 271-279
Non-patent Literature 5: Pursel V G, Hohnson L A. Freezing of boar spermatozoa: Freezing capacity with concentrated semen and a new thawing procedure. J. Anim. Sci. 1975; 40: 99-102

### DISCLOSURE OF THE INVENTION

### Problems which the Invention Tries to Solve

Although various types of transgenic mice into which various genes were introduced and various types of knockout mice in which various types of genes were destructed have been prepared, since the genetic and physiological differences between mouse which is a rodent and human are large, they are not appropriate as models of human in many respects.

Accordingly, an object of the present invention is to provide a transgenic animal having diabetes, which is more suitable as a model of human than rodents, and to provide a preparation method thereof.

### Means for Solving the Problems

The present inventors thought that pigs may be employed as a good model for investigating the influence by eating habits on diabetes and for developing a therapy of diabetes because they are thought to be genetically and physiologically close to human, and also because, in the respect of eating habits, they are omnivorous and eat the same foods as human. The present inventors discovered that a transgenic pig which has diabetes can be prepared by introducing a nucleic acid into a fertilized egg, clonal egg or embryo, the nucleic acid comprising a foreign gene which contains a region encoding dimerization domain of hepatocyte nuclear factor-1α, but does not encode a normal hepatocyte nuclear factor-1α; and developing an individual from the fertilized egg, clonal egg or embryo, thereby completing the present invention.

That is, the present invention provides a method for preparing a transgenic pig with diabetes, the method comprising the steps of introducing a nucleic acid into a fertilized egg, clonal egg or embryo, the nucleic acid comprising a foreign gene which contains a region encoding dimerization domain of hepatocyte nuclear factor-1α, but does not encode a normal hepatocyte nuclear factor-1α, and a promoter located upstream of the foreign gene, which promoter is capable of expressing the foreign gene in a pig cell; and developing an individual from the fertilized egg, clonal egg or embryo. The present invention also provides a transgenic pig prepared by the method according to the present invention, which pig has diabetes, or a progeny thereof which retains the foreign gene and which has diabetes.

### EFFECTS OF THE INVENTION

By the present invention, a transgenic pig with diabetes, into which a mutated HNF-1α gene is introduced, was first provided. Since pigs are genetically and physiologically close to human, the transgenic cloned pig according to the present invention can be used as a model animal suited for the investigation of mechanism of development of diabetes, and for developing a therapeutic method for diabetes. Therefore, it is expected that the present invention will greatly contribute to the research of diabetes of human.

### BRIEF DESCRIPTION OF THE DRAWINGS

FIG. 1 shows a genetic map of CMVPINS-hHNF1αP291fsinsCSVA which is a nucleic acid for preparing a transgenic animal, which was prepared in an Example of the present invention.

FIG. 2 shows a genetic map of PINS-globin-hHNF1αP291fsinsC which is a nucleic acid for preparing a transgenic animal, which was prepared in an Example of the present invention.

### BEST MODE FOR CARRYING OUT THE INVENTION

As described above, in the method for preparing a transgenic pig with diabetes according to the present invention, a foreign gene which contains a region encoding dimerization domain of HNF-1α, but does not encode a normal hepatocyte nuclear factor-1α is introduced into a fertilized egg, clonal egg or embryo (hereinafter also referred to as “fertilized egg or the like” for convenience). The dimerization domain of HNF-1α is located in the 5′-end region thereof. As an example, the base sequence of human HNF-1α is shown in SEQ ID NO:29 in the SEQUENCE LISTING, together with the deduced amino acid sequence encoded thereby. This sequence is known and is described in GenBank Accession No. M57732. In the amino acid sequence shown in SEQ ID NO: 29, the region from the first to 32nd amino acid (hereinafter, the first amino acid, for example, is referred to as “1aa”) is the dimerization domain. Incidentally, in the amino acid sequence shown in SEQ ID NO: 29, 150aa-280aa is Homeobox DNA-binding domain, and 281aa-631aa is transactivation domain. The dimerization domain is the region which forms a homodimer or heterodimer with other HNF-1α or HNF-1β, and if this region exists in the normal state, the HNF-1α can form a homodimer or heterodimer with other HNF-1α or HNF-1β. In the method of the present invention, the foreign gene used for the preparation of the transgenic pig contains a region encoding dimerization domain of HNF-1α, but does not encode a normal HNF-1α. The term “normal HNF-1α” herein means the HNF-1α which forms a homodimer or heterodimer with other HNF-1α or HNF-1β to give a functional transcription factor. Since the foreign gene used in the method of the present invention encodes the dimerization domain but does not encode the normal HNF-1α, it can form a homodimer or heterodimer with other HNF-1α or HNF-1β, but the formed homodimer or heterodimer does not function as a transcription factor.

The mutated HNF-1α gene used in the method of the present invention preferably contains the above-described dimerization domain and Homeobox DNA-binding domain, and the transactivation domain downstream of the Homeobox DNA-binding domain is destructed. Such a destruction can be attained by introducing a frameshift mutation or nonsense mutation at a site downstream of the dimerization domain of HNF-1α, preferably at a site downstream of the Homeobox DNA-binding domain, that is, in the transactivation domain. By introducing such a frameshift mutation or nonsense mutation in an upstream region within the transactivation domain, preferably in the region from first to 100th base (hereinafter, in a base sequence, the first base from the 5′-end is referred to as “1nt”) from the 5′-end of the transactivation domain, more preferably in the region of 1nt-50nt, the structure downstream of the mutated site is lacked or becomes a nonsense structure, so that the transcriptional activity can surely be lost. Such a mutation is preferably a nonsense mutation or a frameshift mutation which yields a stop codon at a site downstream of the mutated site. In the Example below, in the region 888nt-895nt of human HNF-1α gene (SEQ ID NO: 29), which region has consecutive 8 “c”, one additional “c” is inserted to introduce a frameshift mutation which yields a stop codon at 969nt-971nt.

Since the dimerization domain of HNF-1α is well conserved across species, HNF-1α gene of any species may be used as the HNF-1α gene. Since the full length HNF-1α gene originated from human has been sequenced as shown in SEQ ID NO: 29 and the regions of the respective domains have also been clarified as described above, and since it can be easily prepared by PCR using a commercially available cDNA library of hepatic cell, the human HNF-1α gene may preferably be used after introducing the above-described mutation (see Example below).

In the transgenic pig prepared by introducing therein the above-described foreign gene which contains the region encoding the dimerization domain of HNF-1α, but does not encode a normal HNF-1α, the mutated HNF-1α produced by the expression of the introduced foreign gene forms a homodimer or heterodimer with the normal HNF-1α or HNF-1β originated from the pig, and the homodimer or heterodimer containing the mutated HNF-1α does not function as a transcription factor. Therefore, even if normal HNF-1α originated from the pig is produced, it may form a homodimer with the mutated HNF-1α, or the mutated HNF-1α forms a homodimer or heterodimer with normal HNF-1α or HNF-1β, so that the chance for the normal HNF-1α originated from the pig forms a homodimer or heterodimer with the normal HNF-1α or HNF-1β is decreased. As a result, in spite of the fact that normal HNF-1α gene originated from the pig exists, the amount of the functional transcription factor is decreased. Especially, when a strong promoter is used as the promoter for the foreign gene, the mutated HNF-1α originated from the foreign gene is produced in a large amount, so that the probability that the normal HNF-1α originated from the pig forms a functional transcription factor is largely decreased and the amount of the normal transcription factor is largely reduced. As a result, the transgenic pig develops diabetes.

Except for the point that the above-described mutated HNF-1α gene is used as the foreign gene, the transgenic pig according to the present invention can be prepared by a conventional method for preparing transgenic animals. That is, the transgenic pig according to the present invention can be obtained by introducing the nucleic acid containing the promoter regulating the expression of the above-described mutated HNF-1α gene at a site upstream of the mutated HNF-1α gene, into a fertilized egg or the like by the pronuclear injection method or the sperm vector method; and developing an individual from the fertilized egg or the like by returning the embryo to the uterus of a foster mother at an appropriate stage. Although HNF-1α is expressed in the liver, kidney, small intestine and pancreas, to express the mutated HNF-1α in the pancreatic cells, it is preferred to employ a promoter showing a strong promoter activity in pancreatic cells, such as the pig insulin promoter. The pig insulin promoter per se is known (GenBank Accession No. AY044828, AF263916), and is contained in the fragment having the base sequence shown in SEQ ID NO: 17. Although it is difficult to identify the exact location of the promoter, a nucleic acid fragment containing the promoter can be easily obtained. As described below, in the construction of the nucleic acid for preparing the transgenic pig according to the present invention, it is not necessary to isolate the promoter alone, but a nucleic acid fragment containing the promoter can be used. Since a promoter is usually contained in a fragment from the translation start site to the site upstream thereof by about 150 bases, a fragment containing at least this region can be used as the promoter-containing fragment which can regulate the expression of a structural gene located downstream thereof. Thus, although a 674 bp fragment (SEQ ID NO: 17) containing up to a part of the exon 2 of pig insulin is used as the pig insulin promoter-containing fragment in the Example below, it is not necessary to use such a large fragment, but a fragment of the region of about −150 to 0 bp from the transcription start site can be used as the promoter-containing fragment. However, to assure that transcription occurs, it is preferred to include the transcription start site and a short region downstream thereof in the promoter-containing fragment. Promoters other than the pig insulin promoter can also be employed as long as the promoter exhibits promoter activity in pig pancreatic cells.

Although the above-described HNF-1α gene may be ligated to the downstream of the above-described promoter, it is preferred to ligate a rabbit β-globin gene (containing a terminator (polyA) sequence located downstream of exon 3) to the downstream of the promoter, and to insert the mutated HNF-1α gene in the exon 3 thereof. The reason why the exon 3 of the rabbit β-globin is used is that the 3′-untranslated region in the exon 3 has an effect to promote the stability of the mRNA (i.e., to make the mRNA more unlikely to be decomposed) transcribed in the host cell. By inserting the mutated HNF-1α gene in the exon 3 of the rabbit β-globin gene, the exon 2 and a part of the exon 3 of the rabbit β-globin are located between the promoter and the mutated HNF-1α. As a result, an intron exists between the transcription start site and the translation initiation site, which is preferred because the expression of the protein is promoted. The region originated from the rabbit β-globin gene does not contain exon 1 and so the initiation codon does not exist, translation of the region of the rabbit β-globin gene does not occur. The base sequence of the rabbit β-globin gene is also known (GenBank Accession No. V00882), and the gene can be easily prepared by PCR using the rabbit genomic DNA as a template. As the genes having such an action, other than the exon 2 and exon 3 of β-globin gene, 3′ untranslated region of x-globin gene and polyA tail of bovine growth hormone (BGH) are also known, and these may also be used.

The linear nucleic acid fragment in which the rabbit β-globin gene is ligated to the downstream of the promoter and the mutated HNF-1α gene is inserted in the exon 3 thereof can be obtained by, for example, inserting the promoter-containing fragment and the rabbit β-globin gene into a multicloning site of a commercially available cloning vector such as pBluescript series (trade name, produced by Stratagene), inserting the mutated HNF-1α gene in the exon 3 of the rabbit β-globin gene to prepare a circular recombinant vector, and cleaving out a fragment containing the region from the promoter to the terminator of the rabbit β-globin gene (for details, see the Example below). The nucleic acid to be introduced into the fertilized egg or the like is preferably linear in order to increase the probability that the nucleic acid is incorporated into the chromosomal DNA.

Except for the point that the above-described nucleic acid is introduced into the fertilized egg or the like, the transgenic pig according to the present invention can be prepared by a conventional method for preparing transgenic animals. That is, the above-described nucleic acid fragment is introduced into a fertilized egg, clonal egg or embryo by the sperm vector method or pronuclear injection method (see the Example below), both of which are conventional methods. The “clonal egg” herein means an egg obtained by transplanting a nucleus of a somatic cell (in case of somatic cell clone) or of a fertilized egg (in case of fertilized egg clone) into an enucleated recipient egg. The “embryo” herein means an embryo in an optional stage between a unicellular egg and an embryo which can be concepted if returned to a uterus (preferably an embryo in the completely hatched blastocyst stage). However, introducing the gene in the stage of unicellular egg is preferred because the gene is incorporated in all of the cells of the transgenic animal. An individual can be developed, preferably, by growing the egg or embryo into which the gene was introduced up to the morula stage, and returning the resulting embryo to a uterus of an animal.

In the transgenic pig prepared by the method of the present invention, in which the above-described introduced nucleic acid is inserted in the chromosomal DNA, the mutated HNF-1α which cannot function is produced, and this forms a homodimer or heterodimer with the normal HNF-1α or HNF-1β originated from the pig, so that the chance for the normal HNF-1α originated from the pig forms a homodimer or heterodimer with the normal HNF-1α or HNF-1β is decreased. As a result, in spite of the fact that normal HNF-1α gene originated from the pig exists, the amount of the functional transcription factor is decreased and the transgenic pig develops diabetes.

The present invention also provides a transgenic pig prepared by the above-described method according to the present invention, which pig has diabetes, or a progeny thereof which retains the above-described foreign gene and which has diabetes. The term “progeny” herein includes not only the progeny obtained by the normal sexual reproduction, but also the animals cloned from somatic cells, which animals have the same chromosomal DNA as the transgenic animal, and produced by the somatic cell cloning technique. The somatic cell cloning technique has become a conventional method, and concrete procedures are described in detail in the Example below. Since the transgenic animal produced by the production method of the present invention contains the mutated HNF-1α gene in the chromosomal DNA, the animals cloned from the somatic cells using the transgenic animal as a nuclear donor naturally contains the mutated HNF-1α gene.

The transgenic pig according to the present invention has diabetes. Because pigs are thought to be genetically and physiologically close to human, and also because, in the respect of eating habits, they are omnivorous and eat the same foods as human, the transgenic pig according to the present invention is a good model for investigating the influence by eating habits on diabetes and for developing a therapy of diabetes.

The present invention will now be described more concretely by way of an Example thereof. However, the present invention is not restricted to the Example below.

### Example

### 1. Construction of Vector

To express human HNF-1αP291fsinsC by pig insulin promoter, two types of vector, CMVPINS-hHNF-1αP291fsinsCSVA and PINS-globin-hHNF-1αP291fsinsC, were constructed as follows:

(1) Construction of CMVPINS-hHNF-1αP291fsinsCSVA

First, PCR was performed using the First Choice PCR-Ready Human Liver cDNA (Cat#3323, produced by Ambion) as a template. A part of the human HNF-1α cDNA (including from the initiation codon to the stop codon), having a size of 2355 bp was cloned. More concretely, this was carried out as follows: A cDNA fragment (2357 bp) of human HNF-1α was subjected to PCR using First Choice PCR-Ready Human Liver cDNA (produced by Ambion, Cat#3323) as a template dividedly in 3 parts employing the Nco I restriction enzyme recognition sequence (CCATGG) as the boundaries.

The 5′-end portion (856 bp) of the cloned HNF-1α was obtained by nested PCR. First PCR was performed using as primers hHNF-1a-7/hHNF-1a-8: tggcagccgagccatggtttc/gcagcgcaggtcccgggcctg (The forward primer was hHNF-1a-7 whose base sequence was tggcagccgagccatggtttc, and the reverse primer was hHNF-1a-8 whose base sequence was gcagcgcaggtcccgggcctg. A primer set may be hereinafter described as such). The PCR polymerase used was TaKaRa Ex Taq (produced by TAKARA BIO INC.), and the PCR was carried out under the reaction conditions of “94° C./10 min.→(94° C./60 sec.→55° C./60 sec.→70° C./60 sec.) 30 cycles→4° C./∞”. Second PCR was performed using the obtained PCR product as a template. PCR was performed using as primers hHNF-1a-9: gaattctctaaactgagccagctgcagacg to which Eco RI restriction enzyme recognition sequence was added and hHNF-1a-10: ggtaccccatggccagcttgtgccggaagg. The PCR polymerase used was TaKaRa Ex Taq (produced by TAKARA BIO INC.), and the PCR was carried out under the reaction conditions of “94° C./10 min.→(94° C./60 sec.→55° C./60 sec.→70° C./60 sec.) 30 cycles→4° C./∞”. The obtained PCR product was subcloned to pCR2.1-TOPO (Invitrogen), and then sequenced to confirm the sequence.

The central portion (772 bp) of the cloned HNF-1α was obtained by nested PCR. First PCR was performed using as primers hHNF-1a-3/hHNF-1a-6: ggctgggctccaacctcgtcacgg/ggcgctcaggttggtggtgtcggt. The PCR polymerase used was TaKaRa Ex Taq (produced by TAKARA BIO INC.), and the PCR was carried out under the reaction conditions of “94° C./10 min.→(94° C./60 sec.→55° C./60 sec.→70° C./60 sec.) 30 cycles→4° C./∞”. Second PCR was performed using the obtained PCR product as a template. PCR was performed using as primers hHNF-1a-13/hHNF-1a-12: caactggtttgccaaccggcgcaa/catagtctgcgggagcaggcccgt. The PCR polymerase used was TaKaRa Ex Taq (produced by TAKARA BIO INC.), and the PCR was carried out under the reaction conditions of “94° C./10 min.→(94° C./60 sec.→58° C./60 sec.→70° C./60 sec.) 30 cycles→4° C./∞”. The obtained PCR product was subcloned to pCR2.1-TOPO (Invitrogen), and then sequenced to confirm the sequence.

The 3′-end portion (888 bp) of the cloned HNF-1α was obtained by PCR. PCR was performed using as primers hHNF-1a-11: ggtaccccaccatggctcagctgcagagcc and hHNF-1a-2: ggatccacaaggccacgctgatccagggcc to which the Bam HI recognition sequence was added. The PCR polymerase used was TaKaRa Ex Taq (produced by TAKARA BIO INC.), and the PCR was carried out under the reaction conditions of “94° C./10 min.→(94° C./60 sec.→55° C./60 sec.→70° C./60 sec.) 30 cycles →4° C./∞”. The obtained PCR product was subcloned to pCR2.1-TOPO (Invitrogen), and then sequenced to confirm the sequence.

The 3′-end portion subcloned to the pCR2.1-TOPO was cleaved out with Eco RI and Bam HI, and ligated to the Eco RI/Bam HI site in pBluescript SK(−) (trade name, produced by Stratagene). Then the subcloned 5′-end portion was cleaved out with Eco RI, and ligated to the Eco RI site of the pBluescript SK(−) to which the 3′-end portion had been ligated earlier, followed by checking the direction of the ligated fragment. Further, the subcloned central portion was cleaved out with Nco I, and ligated to the Nco I site of the pBluescript SK(−) to which the 5′-end portion and 3′-end portion had been ligated. Finally, the direction of the inserted central portion was checked, thereby completing the cDNA fragment (2357 bp) of human HNF-1α. The base sequence of the obtained cDNA fragment is shown in SEQ ID NO: 11.

To the poly“C” having 8 bp of consecutive “C” located at the site encoding the 291st amino acid (proline) of the cDNA of human HNF-1α, an additional one base of “C” was added by QuickChange Site-Directed Mutagenesis Kit (trade name), thereby constructing hHNF1αP291fsinsC. To the 5′-end of this mutated gene, a 674 bp fragment (SEQ ID NO: 17) containing from the pig insulin promoter to a part of the exon 2 was ligated. To the 3′-end of the mutated gene, 95 bp SV40 early polyadenylation signal (GenBank Accession No. U55762, SEQ ID NO: 20) was ligated. The above-described 674 bp fragment was prepared as follows: That is, the fragment (674 bp) containing pig insulin promoter was obtained by nested PCR. The 1st PCR was performed using as primers pINSprom-1/pINSprom-2: ttggagatgagaagcaggggccag/aggggcaggaggcgcgtccacagg, and using as the template Pig Genomic DNA (Seegene, GDPI2016-1). The PCR polymerase used was TaKaRa Ex Taq (produced by TAKARA BIO INC.), and the PCR was carried out under the reaction conditions of “94° C./180 sec.→(94° C./25 sec.→72° C./180 sec.) 7 cycles→(94° C./25 sec.→67° C./180 sec.) 32 cycles→67° C./420 sec.→4° C./∞”. Second PCR was performed using the obtained PCR product as a template. PCR was performed using as primers pINSprom-3/-4: gaattcaccgccgcagcagcccggggt/gaattcggcggggggtgaggacctggg to each of which Eco RI recognition sequence was added. The PCR polymerase used was TaKaRa Ex Taq (produced by TAKARA BIO INC.), and the PCR was carried out under the reaction conditions of “94° C./180 sec.→(94° C./25 sec.→72° C./180 sec.) 7 cycles→(94° C./25 sec.→67° C./180 sec.) 20 cycles→67° C./420 sec.→4° C./∞”. The obtained PCR product was subcloned to pCR2.1-TOPO (Invitrogen), and then sequenced to confirm the sequence. The SV40 early polyadenylation signal (SEQ ID NO: 20) was prepared as follows: That is, the SV40 early polyadenylation signal (100 bp) was prepared by PCR using as primers BSV40polyA1/XSV40polyA2: ggatccgcagcttataatggttac/tctagaacaaaccacaactagaat to which Bam HI and Xba I recognition sequences were added, respectively, and using as template pEGFP-N1 (trade name, produced by Clontech). The PCR polymerase used was TaKaRa Ex Taq (produced by TAKARA BIO INC.), and the PCR was carried out under the reaction conditions of “94° C./3 min.→(94° C./60 sec.→55° C./60 sec.→70° C./60 sec.) 30 cycles→4° C./∞”. The obtained PCR product was subcloned to pCR2.1-TOPO (Invitrogen), and then sequenced to confirm the sequence.

To convert the Eco RI restriction enzyme recognition sequence (gaattc) to the sequence (atggtt) of translation start site, conversion of the sequence was performed using QuickChange Site-Directed Mutagenesis Kit (trade name). The conversion was carried out using two pairs of primers and the reaction was carried out dividedly in twice, thereby constructing the translation start site. As the primers, mutATG-1/mutATG-2: cctcaccccccgccatattttctaaactgagc/gctcagtttagaaaatatggcggggggtgagg and mutATG-3/mutATG-4: caccccccgccatggtttctaaactgagcc/ggctcagtttagaaaccatggcggggggtg were used. After the conversion of the sequence, the obtained sequence was confirmed by sequencing.

Further, to promote the expression of this mutated gene, an enhancer region (GenBank Accession No. U55762, SEQ ID NO: 23) of human cytomegalovirus immediate early promoter was ligated. This enhancer region was prepared as follows: That is, the enhancer region (419 bp) of the human cytomegalovirus immediate early promoter was prepared by PCR using as primers EcoCMVenS/EcoCMVenA: gaattccgcgttacataacttacgg/gaattccaaaacaaactcccattgac to which Eco RI recognition sequence was added, and using as template pEGFP-N1 (Clontech). The PCR polymerase used was PfuTurbo DNA polymerase (STRATAGENE), and the PCR was carried out under the reaction conditions of “95° C./3 min.→(95° C./30 sec.→54° C./30 sec.→72° C./60 sec.) 30 cycles→72° C./420 sec.→4° C./∞”. The obtained PCR product was subcloned to pCR4Blunt-TOPO (Invitrogen), and then sequenced to confirm the sequence.

Finally, to make the ligated enhancer not influence on other genes, to each of the 5′-end and 3′-end of this vector, a fragment containing insulator sequence (GenBank Accession No. U78775, SEQ ID NO: 28) cloned from chicken β-globin gene was ligated. This insulator sequence was prepared as follows: PCR was performed using two pairs of primers, that is, Eco5insulator-1/EcoInsulator-2: gatatcgggacagcccccccccaaagc/gaattcctcactgactccgtcctggag to which the recognition sequences of Eco RV and Eco RI were added, respectively, and XbaInsulator-1/NotInsulator-2: tctagagggacagcccccccccaaagc/gcggccgcctcactgactccgtcctggag to which the recognition sequences of Xba I and Not I were added, respectively. The PCR polymerase used was TaKaRa Ex Taq (produced by TAKARA BIO INC.), and the PCR was carried out under the reaction conditions of “94° C./180 sec.→(94° C./25 sec.→70° C./180 sec.) 5 cycles→(94° C./25 sec.→65° C./180 sec.) 20 cycles→67° C./420 sec.→4° C./∞”. The obtained PCR product was subcloned to pCR2.1-TOPO (Invitrogen), and then sequenced to confirm the sequence.

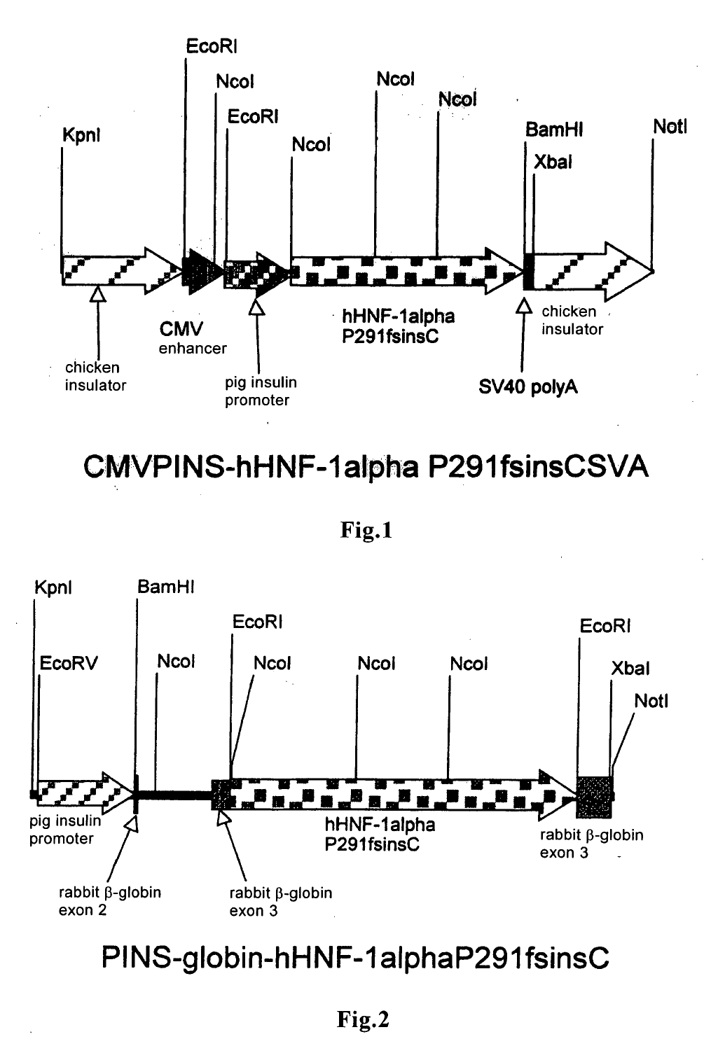

The thus constructed recombinant vector pBS-CMVPINS-hHNF-1αP291fsinsCSVA was digested with Kpn I and Not I to cleave it into two fragments, and the digested product was subjected to agarose gel electrophoresis to separate the two fragments. The Kpn I-Not I fragment containing hHNF-1αP291fsinsC was cut out of the agarose gel and purified with GENECLEAN (trade name) to obtain a nucleic acid (CMVPINS-hHNF1αP291fsinsCSVA) for preparing a transgenic animal. A gene map of CMVPINS-hHNF1αP291fsinsCSVA is shown in FIG. 1. This nucleic acid was diluted to a concentration of 50 ng/μl with TE buffer whose pH had been adjusted to 7.5 and the dilution was cryopreserved, which was used for the microinjection to the pronucleus described below.

(2) Construction of PINS-globin-hHNF1αP291fsinsC

A 864 bp Bam HI-Xba I fragment containing from the exon 2 to the polyadenylation region of rabbit β-globin was inserted into the Bam HI-Xba I site of pBluescript (trade name). The fragment containing the 864 bp Bam HI-Xba I fragment was prepared as follows: First, a fragment (SEQ ID NO: 31) containing the exon 2 to the polyadenylation signal region of rabbit β-globin was prepared. This fragment was prepared by PCR using as template the rabbit genomic DNA, using as forward primer ctgagtgaactgcactgtgac, and using as reverse primer tctagatatgtccttccgagtgaga. The 5′-end of the reverse primer contained an Xba I site added thereto. The PCR polymerase used was PfuTurbo DNA polymerase (STRATAGENE), and the PCR was carried out under the reaction conditions of “94° C./180 sec.→(94° C./25 sec.→72° C./180 sec.) 7 cycles→(94° C./25 sec.→67° C./180 sec.) 35 cycles→67° C./420 sec.→4° C./∞”. The obtained PCR product was subcloned to pCR4Blunt-TOPO (Invitrogen), and then sequenced to confirm the sequence. Finally, the fragment was cleaved out with restriction enzymes Bam HI and Xba I to prepare the 864 bp Bam HI-Xba I fragment of the rabbit β-globin gene.

Then a 665 bp Eco RV-Bam HI fragment (SEQ ID NO: 38) containing from the pig insulin promoter to a part of the exon 2 was inserted into the Eco RV-Bam HI site. This 665 bp Eco RV-Bam HI fragment was prepared by nested PCR as follows: The first PCR was performed using as primers pINSprom-1/pINSprom-2: ttggagatgagaagcaggggccag/aggggcaggaggcgcgtccacagg and using as template Pig Genomic DNA (Seegene, GDPI2016-1). The PCR polymerase used was PfuTurbo DNA polymerase (STRATAGENE), and the PCR was carried out under the reaction conditions of “94° C./180 sec.→(94° C./25 sec.→72° C./180 sec.) 7 cycles→(94° C./25 sec.→67° C./180 sec.) 32 cycles→67° C./420 sec.→4° C./∞”. Second PCR was performed using the obtained PCR product as a template. PCR was performed using as primers pINSprom-5/-6: gatatcaccgccgcagcagcccggggt/ggatcctgaggacctgggggacgggcg to which recognition sequences of Eco RV and Bam HI were added, respectively. The PCR polymerase used was PfuTurbo DNA polymerase (STRATAGENE), and the PCR was carried out under the reaction conditions of “94° C./180 sec.→(94° C./25 sec.→72° C./180 sec.) 7 cycles→(94° C./25 sec.→67° C./180 sec.) 20 cycles→67° C./420 sec.→4° C./∞”. The obtained PCR product was subcloned to pCR4Blunt-TOPO (Invitrogen), and then sequenced to confirm the sequence.

Finally, to the Eco RI site existing in the exon 3 of rabbit β-globin, cDNA (SEQ ID NO: 41) of hHNF1αP291fsinsC to which Eco RI recognition site was added to each of both ends thereof was inserted, and the direction of the cDNA was checked by sequencing, thereby constructing pBS-PINS-globin-hHNF1αP291fsinsC. This cDNA of hHNF1αP291fsinsC to which Eco RI recognition site was added to each of both ends thereof was prepared by PCR using as template the earlier constructed recombinant vector pBS-CMVPINS-hHNF-1αP291fsinsCSVA, and using as primers Eco1HNF1a-16/Eco1HNF1a-17: gaattcccgagccatggtttctaaactgagccagc/gaattcacaaggccacgctgatccagggcc to which Eco RI sequence was added at the 5′-end. The PCR polymerase used was PfuTurbo DNA polymerase (STRATAGENE), and the PCR was carried out under the reaction conditions of “94° C./180 sec.→(94° C./60 sec.→55° C./60 sec.→72° C./180 sec.) 30 cycles→4° C./∞”. The obtained PCR product was subcloned to pCR4Blunt-TOPO (Invitrogen), and then sequenced to confirm the sequence.

The thus constructed recombinant vector pBS-PINS-globin-hHNF1αP291fsinsC was digested with Kpn I and Not I to cleave it into two fragments, and the digested product was subjected to agarose gel electrophoresis to separate the two fragments. The Kpn I-Not I fragment containing hHNF-1αP291fsinsC was cut out of the agarose gel and purified with GENECLEAN (trade name) to obtain a nucleic acid (PINS-globin-hHNF1αP291fsinsC) for preparing a transgenic animal. A gene map of PINS-globin-hHNF1αP291fsinsC is shown in FIG. 2. This nucleic acid was diluted to a concentration of 50 ng/μl with TE buffer whose pH had been adjusted to 7.5 and the dilution was cryopreserved until use.

### 2. Establishment of Nuclear Donor Cell by Sperm Vector Method

Oocytes maturated in improved NCSU23 culture medium (Non-patent Literature 3) or TCM199 culture medium (Non-patent Literature 4) were activated with simple DC pulse (150V/mm, 100 μsec), and then treated with 7.5 μg/ml of cytochalasin B for 3 to 4 hours. The activated oocytes were cultured for 7 days. The concentration of pig sperms cryopreserved in BTS (Non-patent Literature 5) or BF5 (Non-patent Literature 5) solution was adjusted to 2−5×10⁵ sperms, and the sperms were co-cultured with CMVPINS-hHNF1αP291fsinsCSVA DNA (2.5 ng/μl) for 5 minutes. Thereafter, the isolated sperms were injected to IVM oocytes, respectively, with a piezo micromanipulator, and the resulting cells were activated by the electric stimulation as described above. The sperm-injected oocytes were cultured in NCSN23 culture medium for 6 days, and grown to blastocysts. These blastocysts were transplanted to 6 recipient pigs.

35 days after the transplantation of the blastocysts, 4 embryos were obtained from the 6 recipient pigs. Checking the existence of the transgene by PCR and Southern blotting revealed that two of these were transgenic individuals.

The transgenic embryos were minced with a scissors, and after washing with PBS(−), the resultant was centrifuged at 1200 rpm for 5 minutes to separate it into supernatant and precipitate. To this precipitate, 0.25% trypsin-0.01% EDTA was added, and the resulting mixture was incubated at 37° C. for 5 minutes. Thereafter, the resultant was centrifuged at 400 rpm for 5 minutes, and the cells contained in the supernatant were recovered, followed by dispersing the recovered cells in 15% fetal calf serum (FCS)-containing Dulbecco's Modified Eagle's Medium (DMEM). The precipitate was subjected to the above-described operations from the treatment with 0.25% trypsin-0.01% EDTA to obtain a cell dispersion. Finally, the two cell dispersions were centrifuged at 1200 rpm for 5 minutes, and the obtained precipitate was incubated in an incubator under 5% CO₂ at 37.5° C. to establish nuclear donor cells.

### Composition of NCSU23 Culture Medium

NaCl 108.73 mM, KCl 4.78 mM, CaCl₂.2H₂O 1.70 mM, MgSO₄.7H₂O 1.19 mM, NaHCO₃ 25.07 mM, KH₂ PO₄ 1.19 mM, glucose 5.55 mM, glutamine 1.00 mM, taurine 7.00 mM, hypotaurine 5.00 mM, BSA 0.4%, penicillin G 100 IU/L, streptomycin 50 mg/L

### Composition of TCM199 Culture Medium

CaCl₂ (anhydrous) 200.00 mg/L, Fe(NO₃)₃.9H₂O 0.72 mg/L, KCl 400.00 mg/L, mgSO₄ (anhydrous) 97.67 mg/L, NaCl 6800.00 mg/L, NaH₂ PO₄.H₂O 140.00 mg/L, adenosine sulfate 10.00 mg/L, ATP (2Na salt) 1.00 mg/L, adenylic acid 0.20 mg/L, cholesterol 0.20 mg/L, deoxyribose 0.50 mg/L, D-glucose 1000.00 mg/L, glutathione (GSH) 0.05 mg/L, guanine.HCl 0.30 mg/L, hypoxanthine (Na salt) 0.351 mg/L, phenol red 20.00 mg/L, ribose 0.50 mg/L, sodium acetate 50.00 mg/L, thymine 0.30 mg/L, Tween 80 (registered trademark) 20.00 mg/L, uracil 0.30 mg/L, xanthine (Na salt) 0.344 mg/L, DL-alanine 50 mg/L, L-arginine HCl 70.00 mg/L, DL-aspartic acid 60.00 mg/L, L-cystein.HCl.H₂O 0.11 mg/L, L-cystine.2HCl 26.00 mg/L, DL-glutamic acid.H₂O 150.00 mg/L, L-glutamine 100.00 mg/L, glycine 50.00 mg/L, L-histidine.HCl.H₂O 21.88 mg/L, L-hydroxyproline 10.00 mg/L, DL-isoleucine 40.00 mg/L, DL-leucine 120.00 mg/L, L-lysine.HCl 70.00 mg/L, DL-methionine 30.00 mg/L, DL-phenylalanine 50.00 mg/L, L-proline 40.00 mg/L, DL-serine 50.00 mg/L, DL-threonine 60.00 mg/L, DL-tryptophan 20.00 mg/L, L-tyrosine (2Na salt) 57.88 mg/L, DL-valine 50.00 mg/L, ascorbic acid 0.05 mg/L, α-tocopherol phosphate (2Na salt) 0.01 mg/L, d-biotin 0.01 mg/L, calciferol 0.10 mg/L, calcium p-pantothenate 0.01 mg/L, choline hydrochloride 0.50 mg/L, folic acid 0.01 mg/L, i-inositol 0.05 mg/L, menadione 0.01 mg/L, niacin 0.025 mg/L, niacinamide 0.025 mg/L, p-aminobenzoic acid 0.05 mg/L, pyridoxal.HCl 0.025 mg/L, pyridoxine.HCl 0.025 mg/L, riboflavin 0.01 mg/L, thiamine.HCl 0.01 mg/L, vitamin A (acetate) 0.14 mg/L

### Composition of TBS Solution

anhydrous dextrose 3.7 g/100 mL, sodium citrate dihydrate 0.6 g/100 mL, sodium hydrogen carbonate 0.125 g/100 mL, EDTA 2Na 0.125 g/100 mL, potassium chloride 0.075 g/100 mL

### Composition of BF5 Solution

Ter-N-tris(hydroxymethyl)methyl 2 aminoethanesulfonic acid 1.2 g/100 mL, tris(hydroxymethyl)aminoethane 0.2 g/100 mL, anhydrous dextrose 3.2 g/100 mL, yolk 20 mL/100 mL, Orbus BS paste 0.5 mL/100 mL

### 3. Somatic Cell Nuclear Transplantation

In a meat factory, an ovary was placed in Dulbecco's PBS (PBS(−)—PVA) supplemented with 75 μg/ml penicillin G, 50 μg/ml streptomycin sulfate and 0.1% polyvinyl alcohol, and transported in the condition of being warmed at 24° C. to 30° C. The transported ovary was washed with 0.2% cetyltrimethylammonium bromide (CETAB) and then washed three times with (PBS(−)—PVA), and incubated at 38.5° C. in an incubator until use. Thereafter, under the condition of being warmed at 38.5° C., using a 20 G injection needle and a 5-ml syringe, eggs were aspirated from ovarian follicles with a diameter of 3 mm to 6 mm together with follicular fluid. The obtained follicular fluid was centrifuged at 800 rpm for 2 minutes to precipitate the eggs. The obtained eggs were dispersed into TL-Hepes-PVP, and cumulus-egg complexes having a number of attached cumulus cells and having an egg whose cytoplasm was normal were selected under the microscope, followed by culturing the selected complexes in NCSU23 culture medium supplemented with 0.6 mM cystein, 10 μg/ml epidermal growth factor (EGF), 10% pig follicular fluid, 70 μg/ml penicillin G, 50 μg/ml streptomycin sulfate, 10 IU/ml equine chorionic gonadotropin (eCG) and 10 IU/ml human chorionic gonadotropin (hCG), in an incubator at 38.5° C. 22 hours after the beginning of the in vitro maturation culture, the cells were transferred to NCSU23 from which the hormones had been removed, and cultured for another 22 hours.

The eggs after completion of the in vitro maturation culture were treated with 0.01% hyaluronidase, and the cumulus cells and the granular layer cells were removed by pipetting in a drop of TL-Hepes-PVP. Then only the eggs which extruded the first polar body, that is the feature of maturated eggs, were selected, and the selected eggs were used as recipient eggs. In this case, dead eggs and eggs whose cytoplasm had an irregular shape were excluded.

The recipient eggs were enucleated by aspirating the cytoplasm in the vicinity of the first polar body by micromanipulation in TL-Hepes-PVP supplemented with 7.5 μg/ml cytochalasin B and 10% fetal calf serum (FCS) using a pipette having a keen edge and having an aperture diameter of 30 μm. The enucleated eggs were stained by being placed in a TL-Hepes-PVP drop supplemented with 5 μg/ml Hoechst 33342 for 5 minutes, and whether enucleation was succeeded or not was confirmed with a fluorescence microscope.

Each of the recipient eggs was put on standby in a TL-Hepes-PVP drop supplemented with 10% fetal calf serum (FCS), and each of the nuclear donor cells was, after being peeled off with 0.1% trypsin-0.01% EDTA, was put on standby in a NCSU23-Hepes (NCSU23 containing 21 mM Hepes) drop supplemented with 10% fetal calf serum (FCS). The nuclear donor cell was inserted into the perivitelline space of the recipient egg through the hole in the zona pellucida formed during the enucleation, by micromanipulation using a pipette having a keen edge and having an aperture diameter of 30 μm. The egg to which the cell was inserted was placed in a drop of mannitol solution (0.3 M mannitol supplemented with 50 μM calcium chloride, 100 μM magnesium chloride and 0.01% polyvinyl alcohol) for cell fusion, and was clamped with electrodes such that the contact face between the egg and the cell inserted into the perivitelline space was perpendicular to the electric current, followed by carrying out cell fusion with a cell fusion apparatus (SSH-1 produced by Shimadzu Corporation). The cell fusion was carried out under the conditions of “alternating current 1 MHz, 5V, 5 sec, direct current 200V/mm, 10 μsec, once”.

1 to 1.5 hours after the cell fusion, activation by electric stimulation was performed. The activation was carried out by forming a drop of mannitol solution (0.3 M mannitol supplemented with 50 μM calcium chloride, 100 μM magnesium chloride and 0.01% polyvinyl alcohol) between electrodes (width: 1 mm) placed in parallel each other on a slide glass, aligning the embryos succeeded in cell fusion in a row under the microscope, and electrically stimulating the embryos under the conditions of “direct current 100V/mm, 100 μsec, once” with a cell fusion apparatus (SSH-1 produced by Shimadzu Corporation). Since the activated egg extrudes the second polar body, they were transferred to NCSU23 supplemented with 5 μg/ml cytochalasin B and 4 mg/ml bovine serum albumin (BSA) before extrusion of the second polar body and cultured for 3 hours, thereby carrying out a polyploidization treatment.

Each of the embryos after the activation and the polyploidization treatment was cultured in vitro in NCSU23 supplemented with 4 mg/ml bovine serum albumin (BSA) in an incubator under 5% CO₂ at 38.5° C. 96 hours after the beginning of the in vitro culture, fetal calf serum (FCS) was added to the drops in which the embryos were cultured to a concentration of 10%. 168 hours after the beginning of the in vitro culture, each embryo which developed to blastocyst was transplanted to a pig. Four months after the transplantation of the blastocyst, transgenic cloned pigs were obtained from recipient pigs.

Eight transgenic cloned pigs were born from 4 recipient pigs, and five of them were born normally (three were born dead). The body weights of the born transgenic cloned pigs were in the range of 610 g to 810 g, which were smaller than those (960 g to 1790 g) of the control group to which the foreign gene was not introduced. Among the normally born 5 pigs, the individual having the largest body survived until 20 days of age, which survival was the longest.

The body weight of this individual at 10 days of age was 2660 g, so that the body weight gain was similar to those of the individuals in the control group. However, the body weight gain thereafter was small. Further, it showed a higher blood glucose level (200 to 250 mg/dl) than those of the individuals in the control group. The body weight at the time of death at 20 days of age was 2880 g, so that the body weight gain after 10 days of age was small.

Pathologic specimens of this transgenic cloned pig were prepared and observed. As a result, it was observed that the degree of pyknosis and denaturation of cytoplasm of the Langerhans' islands-constituting cells in the pancreas was higher than those of the individuals in the control group. By immunostaining with an anti-insulin antibody, the number of positive cells and small groups of positive cells was larger than those in the individuals in the control group, and the number of the clear and orderly aligned positive cells was small, which were observed in the individuals in the control group. These observation results indicate that the construction of the Langerhans' islands in the transgenic cloned pig was insufficient.

From these results, it was judged that in this transgenic cloned pig, insulin was short due to the insufficient construction of Langerhans' islands, and this transgenic cloned pig died of illness due to diabetes.

1. A method for preparing a transgenic pig with diabetes, said method comprising the steps of: introducing a nucleic acid into a fertilized egg, clonal egg or embryo, said nucleic acid comprising a foreign gene which contains a region encoding dimerization domain of hepatocyte nuclear factor-1α, but does not encode a normal hepatocyte nuclear factor-1α, and a promoter located upstream of said foreign gene, which promoter is capable of expressing said foreign gene in a pig cell; and developing an individual from said fertilized egg, clonal egg or embryo.

2. The method according to claim 1, wherein said foreign gene is a mutated hepatocyte nuclear factor-1α, in which a frameshift mutation or nonsense mutation is introduced at a site downstream of said dimerization domain of hepatocyte nuclear factor-1α.

3. The method according to claim 1 or 2, wherein said foreign gene comprises said region encoding the dimerization domain of hepatocyte nuclear factor-1α, and a region encoding Homebox DNA-binding domain.

4. The method according to any one of claims 1 or 2, wherein said promoter is pig insulin promoter.

5. A transgenic pig prepared by the method according to any one of claims 1 or 2, which pig has diabetes, or a progeny thereof which retains said foreign gene and which has diabetes.
